(19) United States
(12) Patent Application Publication
(10) Pub. No.: US 2022/0017925 A1
(43) Pub. Date: Jan. 20, 2022

(54) COMPOSITION COMPRISING AT LEAST ONE NANOBOMB SUITABLE FOR ALTERING A BIOLOGICAL BARRIER

(71) Applicant: Universiteit Gent, Gent (BE)

(72) Inventors: Kevin Braeckmans, Lokeren (BE); Juan Fraire, Brussel (BE); Stefaan De Smedt, Mariakerke (BE)

(21) Appl. No.: 17/298,884

(22) PCT Filed: Dec. 16, 2019
(86) PCT No.: PCT/EP2019/085391

§ 371 Date: Jun. 1, 2021

(30) Foreign Application Priority Data
Dec. 18, 2018 (EP) 18213548.3

(51) Int. Cl. C12N 15/89; A61K 41/00; A61K 48/00; A61K 47/69; C12N 15/88

(52) U.S. Cl. CPC C12N 15/895; A61K 41/0042; A61K 48/0041; C12N 15/88; A61K 47/6925; A61K 47/6915; A61K 48/0083

(57) ABSTRACT

A composition comprising at least one nanobomb comprising at least one first particle and at least one second particle in close proximity to the first particle. The at least one first particle is able to absorb electromagnetic radiation so as to generate a vapor bubble. The generation of the vapor bubble causes the at least one second particle to be propelled over a distance D. The composition is suitable to alter a biological barrier, in particular, for deforming, permeabilizing or perforating a biological barrier. A method to alter biological barriers is also disclosed.

### CROSS-REFERENCE TO RELATED APPLICATIONS

This application is a national phase entry under 35 U.S.C. § 371 of International Patent Application PCT/EP2019/085391, filed Dec. 16, 2019, designating the United States of America and published as International Patent Publication WO 2020/127076 A1 on Jun. 25, 2020, which claims the benefit under Article 8 of the Patent Cooperation Treaty to European Patent Application Serial No. 18213548.3, filed Dec. 18, 2018.

### TECHNICAL FIELD

A composition comprising at least one nanobomb suitable for altering a biological barrier and, in particular, suitable for deforming, permeabilizing or perforating a biological barrier. More specifically, a composition suitable for drug delivery, cell therapy, immunotherapy, gene therapy and transfection of cells is disclosed. The disclosure further relates to the use of a composition in a method for altering biological barriers, in particular, in a method for deforming, permeabilizing or perforating biological barriers.

### BACKGROUND

Delivering exogenous compounds into cells is a ubiquitous requirement in biomedical applications, whether it is for the creation of mutant cell lines in biology, drug screening of biologicals in pharmacy, or labeling of cells for imaging. Regardless of the specific application, the common challenge is to overcome the cell membrane, which represents a major obstacle for most macromolecules and nanoparticles.

Physical methods for the delivery of compounds into cells have attracted considerable interest. Such methods have in common that the permeability of the cell membrane is increased, allowing passage of compounds, such as molecules or nanoparticles, across the cell membrane.

A known physical method for the delivery of compounds into cells is electroporation. By electroporation, small pores are formed in the cell membrane through the application of high-voltage electrical pulses. Although this technique has been applied for decades, it has major drawbacks. One drawback is that the technique induces high cytotoxicity.

An alternative and more recent technique for the delivery of compounds into cells is laser-induced photoporation. In its original form, pores are created in the cell membrane by high-intensity femtosecond (fs) laser pulses focused precisely with respect to the cell membrane. The throughput of this process can be improved considerably by using sensitizing nanoparticles such as gold nanoparticles. The nanoparticles are first adsorbed onto the cell membrane and subsequently irradiated with wide field (i.e., unfocused) laser irradiation. Upon absorption of the laser light, the cell membrane becomes permeabilized through localized photothermal effects, such as local heating or the formation of laser-induced water vapor nanobubbles (VNB).

Although laser-induced photoporation is a promising technique, it has some drawbacks. As the size of the pores that can be generated in the cell membrane is limited, there is a size limit of the compounds that can be delivered into the cells as well. Especially large macromolecules, such as plasmid DNA, have a very low delivery efficiency. Furthermore, laser-induced photoporation is not suitable to permeabilize some types of cells, for example, cells with a strong outer cell wall such as plant cells. Additionally, plasmonic nanoparticles, such as gold nanoparticles, tend to fragment into tiny pieces upon intensity laser illumination as used for forming VNB. Reportedly, such small gold nanoparticles have the potential to be genotoxic when internalized into cells. Considering that the photoporation method requires close contact between the plasmonic nanoparticles and the cells, there might be a nanotoxicological concern to use photoporation, for example, for transfecting cells to be used in cell therapies.

### BRIEF SUMMARY

A composition is provided comprising at least one nanobomb suitable for altering a biological barrier, for example, a cellular membrane, avoiding the drawbacks of the compositions known in the art.

A composition is also provided comprising at least one nanobomb suitable for deforming, permeabilizing or perforating a biological barrier, for example, a cellular membrane, avoiding the drawbacks of the compositions known in the art.

A composition suitable for drug delivery, cell therapy, immunotherapy, gene therapy and transfection of cells is also provided.

A composition is also provided comprising at least one nanobomb comprising at least one first particle and at least one second particle, whereby the first particle(s) may generate a vapor bubble and the second particle(s) may function as a nanoprojectile.

A composition is further provided that is suitable for altering a biological barrier avoiding close contact or direct contact between the first particles of the composition and the biological barrier, for example, not requiring close or direct contact between the first particles and the cellular membrane.

A composition is further provided that allows a biological barrier, for example, a cellular membrane, with pores having a size between 1 nm and 1000 nm, for example, between 10 nm and 1000 nm, allowing relatively large compounds to cross the biological barrier, for example, the cellular membrane.

A composition is further provided that comprises at least one nanobomb having functionalized second particles, for example, drug-loaded nanoprojectiles.

A method is also provided to alter biological barriers, for example, cellular membranes, in particular, suitable for drug delivery, cell therapy, immunotherapy, gene therapy and transfection of cells.

Furthermore, the use of a composition comprising nanobombs for the intracellular delivery of exogenous materials is provided.

According to a first aspect of the present disclosure, a composition comprising at least one nanobomb, preferably comprising multiple nanobombs, is provided. The at least one nanobomb comprises n first particles and m second particles, with each of n and m being at least one. At least one of the m second particles is in close proximity to at least one of the n first particles. Preferably, a first particle of a nanobomb is surrounded by one or more, preferably by more than one, second particles in close proximity to this first particle. The first particle that is surrounded by the one or more second particles is thereby referred to as the central first particle. The second particle or second particles surrounding such first particle is/are referred to as the surrounding particle(s).

The second particle or second particles preferably have a size equal or smaller than the size of the first particle or first particles. More preferably, the second particle or second particles have a size smaller than the first particle or first particles. The size of the second particle or second particles is, for example, a factor 2 smaller than the size of the first particles, a factor 10 smaller than the size of the first particles or a factor 20 smaller than the size of the first particles.

The first particles are able to absorb electromagnetic radiation so as to generate a vapor bubble, for example, a vapor microbubble or vapor nanobubble, whereby the generation of the vapor bubble causes the at least one second particle (i.e., the at least one of the m second particles that is in close proximity to at least one of the n first particles) to be propelled over a distance D away from the at least one first particle, with the distance D being larger than 0.01 μm, the distance D being defined as the displacement of the at least one second particle as a consequence of the process of generating vapor bubbles.

For the purpose of this disclosure, the term “in close proximity to” or “being in close proximity to” is defined as either being in contact with or being positioned at a distance d smaller than 1 μm, with the distance d being the closest distance between the outer surface of the at least one second particle and the outer surface of the at least one first particle. For the purpose of this disclosure, the term “in close proximity to” or “being in close proximity to” is used with reference to neighboring or surrounding particles. The term “in close proximity to” or “being in close proximity to” does not include being incorporated or integrated in. Consequently, a second particle in close proximity to a first particle is located next to this first particle and is not incorporated nor integrated in this first particle. Similarly, a first particle in close proximity to a second particle is located next to this second particle and is not incorporated nor integrated in this second particle.

The distance d can be zero or non-zero. Preferably, the distance d ranges between 0 nm and 500 nm, for example, between 0 nm and 50 nm or between 0 nm and 10 nm, for example, 0.1 nm, 0.5 nm, 1 nm or 5 nm.

The term “being in contact with” includes any type of contact and includes, amongst others, being connected, being attached and touching one another. In particular, the term “being in contact with” includes bioconjugation, complexation, electrostatic connection, physisorption, chemical connection as, for example, chemical connection by one or more covalent bonds. Preferred ways of being in contact comprise connection by covalent bond(s), physisorption, electrostatic connection and bioconjugation.

In some embodiments, the n first particles and m second particles of a nanobomb are held together by a matrix material or by a surrounding or partially surrounding physical barrier or shell. The n first particles and the m second particles of a nanobomb are, for example, encapsulated within the matrix material of an enclosing material, such as a polymer material to form a matrix that holds first and second particles together in close proximity. Alternatively or additionally, the n first particles and the m second particles are held together by a surrounding shell, such as a polymer shell, to form a microcontainer or nanocontainer with first and second particles in close proximity inside. It is clear that a matrix material or a surrounding or partially surrounding physical barrier or shell may comprise one nanobomb or a plurality of nanobombs.

As described above, the first particle or first particles of a nanobomb according to the disclosure is/are able to absorb electromagnetic radiation so as to generate a vapor bubble preferably in or from a surrounding medium. This means that upon irradiation of the nanobomb, in particular, upon irradiation of the first particle of the nanobombs, a vapor bubble is generated. Preferably, the vapor bubble is generated by evaporation of the medium surrounding the first particle, the vapor bubble is, for example, generated by evaporation of water surrounding the first particle. Alternatively, a vapor bubble is generated by evaporation or partial evaporation of the first particle. Furthermore, a vapor bubble can be generated by a combination of evaporation of the medium surrounding the first particle and evaporation or partial evaporation of the first particle. Upon irradiation, preferably with short pulsed laser light of sufficient intensity, the temperature of the first particle rapidly increases (usually to several hundred degrees) due to heat confinement and the medium surrounding the first particle and/or the material of the first particle evaporating quickly. This results in the generation of a vapor bubble that is quickly expanding around the particle surface, possibly followed by collapsing. The generation of such a vapor bubble propels the second particle or particles in close proximity over a distance D in a direction away from the first particle.

It is stressed that a vapor bubble referred to according to the present disclosure is caused by evaporation of the medium surrounding the material of the nanobomb, in particular, the medium surrounding the first particle(s). The material of the nanobomb, in particular, the material of the first particle(s), is thereby not evaporated.

The second particle(s) of a nanobomb according to the disclosure is/are preferably adapted to alter at least partially a biological barrier once propelled upon generation of the vapor bubble. More particularly, the second particle(s) of a nanobomb according to the present disclosure is/are adapted to deform a biological barrier at least partially once propelled upon the generation of the vapor bubble. In preferred embodiments, the second particle(s) of a nanobomb according to the disclosure is/are adapted to permeabilize a biological barrier once propelled upon the generation of the vapor bubble. In particular preferred embodiments, the second particle(s) of a nanobomb according to the present disclosure is/are adapted to perforate a biological barrier once propelled upon the generation of the vapor bubble.

Biological barriers include, but are not limited to, cellular membranes or barriers as, for example, cell membranes or cell walls of eukaryotic and prokaryotic cells, intracellular membranes, such as endosomal membranes, nuclear envelopes, mitochondrial membranes, etc. Biological barriers also include, but are not limited to, multicellular tissues, such as mucosa, blood-brain barriers, blood-retina barriers, microbial biofilms, etc. Biological barriers furthermore include, but are not limited to, extracellular matrices, such as mucus, vitreous humor, basal lamina, biofilm matrices, etc.

The terms “alter,” “altering,” or “alteration” refer to any way to change one or more properties of a biological barrier at least partially, for example, at least locally. Altering includes, but is not limited to, inducing a local change in the biological barrier's composition by adding, removing, destroying or reorganizing constituents through the action of the nanobombs, in particular, of the second particle or second particles of the nanobombs. For instance, the second particle or second particles of a nanobomb may be adapted to change one or more physicochemical properties of a biological barrier, such as its viscosity, porosity, density, rigidity, elasticity, etc. In another example, the propelling of the second particle or second particles may result in a local destruction or rearrangement of barrier constituents, resulting in a change of the composition and/or physicochemical properties of the barrier. In yet another example, the second particle or second particles of a nanobomb may be adapted to be composed of or to release active compounds, for example, active pharmaceutical compounds, that may induce (bio)chemical changes to the barrier.

The terms “deform,” “deforming,” and “deformation” refer to any way to alter the spatial organization or structure of a biological barrier at least partially, for example, at least locally. Examples of deforming comprise providing the biological barrier with indentations or invaginations.

The terms “permeabilize,” “permeabilizing,” and “permeabilization” refer to any way to alter the permeability of a biological barrier at least partially, for example, at least locally. Examples of permeabilizing comprise altering the barrier composition or structure so that it becomes more permeable to one or more types of molecules, particles or nanoparticles.

The terms “perforate,” “perforating” or “perforation” refer to any way to provide a biological barrier at least partially, for example, at least locally, with one or more openings, holes or pores. By perforating a biological barrier, openings are created into the barrier allowing the transport of compounds, such as molecules, particles or nanoparticles, across or into that barrier.

For the purpose of this disclosure, the terms “perforate,” “perforating,” “perforation,” and the terms “permeabilize,” “permeabilizing,” “permeabilization” are interchangeably used. Similarly, for the purpose of this disclosure, the terms “opening,” “hole,” and “pore” are interchangeably used.

Recent publications of the ballistic response of lipid membranes showed that nanoprojectiles impacting on lipid membranes can follow three different pathways depending on the initial velocity, the size and the density of the projectile. These pathways are rebounding, localized-deformation penetration and tube-formation penetration. Although applicant does not want to be bound by any theory, the terms “deforming,” “permeabilizing,” and “perforating” within the context of this application include the generation of pores through localized-deformation penetration as well as local changes of the membrane composition by invagination of the membrane due to, for instance, tube formation.

As the at least one second particle of a nanobomb is propelled over a distance D upon generation of the vapor bubble, the biological barrier(s) is/are preferably located at a distance smaller than or equal to D. Therefore, the composition comprising the at least one nanobomb is preferably introduced in the proximity of the biological barrier to be altered, deformed, permeabilized or perforated. In particular, the composition comprising the at least one nanobomb is preferably introduced in the proximity of the cells to be altered, deformed, permeabilized or perforated. Direct contact between the composition and the biological barriers, i.e., between the nanobombs and the biological barriers is possible but not necessarily required. In particular, direct contact between the composition and the cells, i.e., between the nanobombs and the cells, is possible but not necessarily required.

Compared to techniques known in the art, it is an important advantage of the disclosure that the composition according to the present disclosure does not depend on direct contact between the composition and the biological barrier to be altered, deformed, permeabilized or perforated. In particular, by using the composition according to the present disclosure, direct contact is not necessarily needed between plasmonic nanoparticles and the biological barriers to be altered, deformed, permeabilized or perforated. Consequently, potential risks due to potential toxicity of plasmonic nanoparticles can be avoided or substantially reduced.

The at least one second particle of a nanobomb according to the disclosure is preferably adapted to be propelled over a distance D, with the distance D ranging between 0.01 and 1000 μm, more preferably between 0.1 and 100 μm, between 0.5 and 20 μm or between 1 and 20 μm.

During the expansion of the vapor bubble, the vapor bubble boundary is moving outwards with a velocity vₑₓₚₐₙₛᵢₒₙ till the vapor bubble reaches its maximum size at time tₘₐₓ. The velocity vₑₓₚₐₙₛᵢₒₙ generally ranges between 0.1 and 100 m/s as, for example, between 0.5 and 20 m/s. The time tₘₐₓ generally ranges between 10 ns and 1 μs and, for example, between 50 ns and 200 ns. As the vapor bubble is expanding with a velocity vₑₓₚₐₙₛᵢₒₙ and as the second particles are pushed forward by the expanding vapor bubble, one expects that the maximum initial velocity v₀ of the second particles is equal to vₑₓₚₐₙₛᵢₒₙ. Not intending to be bound by any theory, one expects that the distance D over which a second particle is propelled, is, amongst others, depending on the density of the second particle, the weight of the second particle, the dimensions of the second particle and the initial velocity v₀ of the particle, the fluid density p and the fluid's dynamic viscosity η. The maximum distance over which a second particle is propelled is estimated for a 100 nm spherical nanoprojectile having an initial velocity of 10 m/s in water. The estimated maximum distance is referred to as D_{max est}. The corresponding Reynolds number is Rₑ≈1, as calculated from Rₑ=ρv₀d_{2P}/η, with ρ the fluid density, v₀ the initial velocity of the second particle, d_{2P} the diameter of the second particle and η the fluid's dynamic viscosity. In this regime of low Reynold numbers, the drag force experienced by the nanoprojectile can be approximated by the Stokes drag force F_{d}=−3πηvd₂ₚ. From Newton's equation of motion, it then follows that the estimated distance Dₑₛₜ travelled as a function of time t is

$${D}_{e s t} = \frac{m {v}_{0}}{3 π η {d}_{2 P}} ( 1 - {e}^{- 3 π η {d}_{2 P} t / m} ) ,$$

with m the mass of the second particle. In the limit for large t, it follows that the second particle will travel an estimated maximum distance

$${D}_{max est} = \frac{m {v}_{0}}{3 π η {d}_{2 P}} .$$

Taking, for example, a second particle composed of polystyrene with a mass density of 1.05 kg/dm³, it follows that the second particle will travel no further than D_{max est}≈0.006 nm. From this estimation, one can derive that the maximum distance D_{max est} generally will be substantially smaller than 1 nm.

Surprisingly, it was found that the second particles of a nanobomb according to the present disclosure are propelled over a distance D, which is considerably larger than expected, on the above theoretical grounds, more particularly, over a distance D larger than 0.1 μm, which is over a distance at least 10,000 times larger than expected.

In preferred embodiments, the distance D is larger than 1 μm (this is 100,000 times larger than expected), larger than 10 μm (this is 1,000,000 times larger than expected) or even 100 μm (this is 10,000,000 times larger than theoretically expected.

Preferably, the composition according to the present disclosure comprises multiple nanobombs. The concentration of nanobombs in a composition depends, for example, on the application. A composition according to the present disclosure used to alter cell membranes has, for example, a concentration of nanobombs ranging between 10⁵ and 10¹² nanobombs/mL and, more preferably, between 10⁷ and 10¹⁰ nanobombs/mL, as, for example, 5×10⁷ nanobombs/mL, 5×10⁸ nanobombs/mL or 5×10⁹ nanobombs/mL.

As mentioned above, a nanobomb according to the disclosure comprises n first particles and m second particles, with each of n and m being at least 1, i.e., with each of n and m being larger than or equal to 1. At least one of the m second particles of a nanobomb is in close proximity to at least one of the n first particles. In a preferred embodiment, the number of second particles m is larger than the number of first particles n.

In a preferred embodiment, a nanobomb comprises one first particle (n=1) and one second particle (m=1).

In other preferred embodiments, a nanobomb comprises one first particle (n=1) and m second particles with m being larger than 1.

In further preferred embodiments, a nanobomb comprises m first particles and n second particles, with m and n being larger than 1, m can be larger than or equal to n, preferably m is larger than n.

Preferably, a first particle of a nanobomb has more than one second particle, for example, p second particles in close proximity to this first particle, with p being at least 2. The p second particles are thereby surrounding the first particle and are positioned at a distance d smaller than 1 μm from the first particle, with d being the closest distance between the outer surface of the at least one second particle and the outer surface of the first particle.

Preferably, the majority of the n first particles comprises at least p second particles in close proximity with a first particle, with p being at least 2. With the majority of the n first particles is meant at least 50%, at least 60%, at least 80% or at least 90% of the first particles.

The maximum number p of second particles per first particle depends, amongst others, upon the configuration of the first and second particles in the nanobombs, upon the size of the first particles and/or upon the size of the second particles.

In case a first particle is surrounded by one layer of second particles, the maximum number of second particles per first particle is limited by the maximum loading capacity of the first particle, i.e., the maximum of second particles that can be positioned around the first particle so that the second particles are in close proximity to the first particle. It is clear that the maximum loading capacity depends, amongst others, on the size of the first particle, the size of the second particles and the connection strategy. Preferably, the number of second particles surrounding the first particle ranges between 10% and 100% of the maximum loading capacity of the first particle; more preferably, the number of second particles surrounding the first particles ranges between 50% and 100% and is, for example, 60%, 70%, 80% or 90% of the maximum loading capacity of the first particle.

In case a plurality of first particles (n>1), for example, first particles in contact with each other or connected to each other, is surrounded by one layer of second particles, the maximum number of second particles is, for example, limited by the maximum loading capacity of the plurality of first particles, i.e., the maximum of second particles that can be positioned around the plurality of first particles so that the second particles are in close proximity to a first particle. It is clear that the maximum loading capacity depends, amongst others, on the size of the first particle, their connection strategy, the size of the second particle and their connection strategy. Preferably, n ranges between 10% and 100% of the maximum loading capacity of a plurality of the first particles; more preferably, n ranges between 50% and 100% and is, for example, 60%, 70%, 80% or 90% or the maximum loading capacity of the plurality of first particles.

In case a first particle or a plurality of first particles is surrounded by more than one layer of second particles, for example, by 2 layers or 3 layers of second particles, the number p of second particles per first particle or per plurality of first particles can be higher than the maximum loading capacity of the first particle.

In case a nanobomb comprises more than one first particle (n>1), the first particles can be either in contact with each other or connected to each other or not in contact with each other or not connected to each other. The first particles form, for example, a cluster of first particles. Preferred ways to be in contact or to be connected include bioconjugation, complexation, electrostatic connection, physisorption, chemical connection as, for example, chemical connection by one or more covalent bonds. Preferred ways of nanobombs comprising more than one first particle not in contact or not connected to each other comprise nanobombs comprising first particles in a matrix material or surrounded by a physical shell such as a polymer shell to form a micro or nanomatrix or a micro or nanocontainer, respectively. The number of first particles and the number of second particles present in a matrix or container depends, amongst others, upon the size of the first and second particles and/or upon the size and/or composition of the matrix or the container. A larger container comprises, for instance, more first and second particles as compared to a smaller one. Also, the number of nanobombs present in a matrix or container depends, amongst others, upon the size of the first and second particles and/or upon the size and/or composition of the matrix or the container.

In case a nanobomb comprises more than one first particle (n>1), the first particles of the nanobomb can be the same or can be different. In case a nanobomb comprises different first particles, the first particles may, for example, have a different composition and/or a different size.

In the composition, the number of first particles per nanobomb can be constant or can vary. Preferably, the number of first particles per nanobomb is constant or substantially constant.

In case a nanobomb comprises more than one second particle (m>1), the second particles of the nanobomb can be the same or can be different. In case the nanobomb comprises different second particles, the second particles may, for example, have a different composition and/or a different size.

In the composition, the number of second particles per nanobomb can be constant or can vary. Preferably, the number of second particles per nanobomb is constant or substantially constant.

It is clear that the nanobombs should not have to be identical in the composition. As mentioned above, the number of first particles per nanobomb as well as the number of second particles per nanobomb may vary in the composition. Furthermore, different nanobombs in a composition may comprise first particles that vary in composition and/or in size and/or different nanobombs in a composition may comprise second particles that vary in composition and/or in size. Also, the distance d between the first particle and the first particle and the connection strategy between first and second particles may vary within the composition.

As first particle, any particle that is adapted to cause a vapor bubble upon electromagnetic irradiation can be considered. Preferably, the first particles comprise biocompatible particles. More preferably, the particles comprise clinically approved particles.

The first particles preferably have an average particle size between 0.01 μm and 10 μm, more preferably, between 0.02 μm and 7 μm as, for example, between 0.1 μm and 5 μm, for example, 0.03 μm, 0.1 μm, 0.2 μm, 0.5 μm, 1 μm, 2 μm or 5 μm. The average particle size is preferably determined by a high resolution imaging technique such as transmission or scanning electron microscopy (TEM or SEM) or atomic force microscopy (AFM).

Preferred first particles comprise a metal, a metal oxide, carbon or a carbon-based material, a light-absorbing material or a material loaded or functionalized with one or more light-absorbing material(s) or compound(s) or a combination thereof. First particles may, for example, comprise a combination of metals, a combination of metal oxides, a combination of carbon-based materials, a combination of light-absorbing materials or a combination of materials loaded or functionalized with one or more light-absorbing materials or compounds or any other combination.

Examples of metals include gold, silver, platinum, palladium, copper and alloys thereof. Preferred metals include gold, silver and alloys thereof.

Examples of metal oxides include iron oxide, titanium oxide, zirconium oxide, cerium oxide, zinc oxide and magnesium oxide.

Examples of carbon or a carbon-based material include graphene or graphene oxide.

Examples of a light-absorbing compound include synthetic organic or inorganic absorbers as well as naturally occurring absorbers or derivatives thereof, for example, light-absorbing dye molecules such as indocyanine green, inorganic quantum dots (having low fluorescence quantum yield), naturally occurring light absorbers like pigments (such as melanin, rhodopsin, photopsins or iodopsin) and synthetic analogs like polydopamine, or photosensitizers used in photodynamic therapy.

Preferred first particles include metal particles, metal oxide particles, carbon or carbon-based particles, particles comprising one or more light-absorbing compounds or particles loaded or functionalized with one or more light-absorbing compounds.

Examples of metal particles include gold particles, silver particles, platinum particles, palladium particles, copper particles and alloys thereof. Preferred metal particles include gold particles, silver particles and alloys thereof.

Examples of metal oxide particles include iron oxide, titanium oxide, zirconium oxide, cerium oxide, zinc oxide and magnesium oxide.

Examples of carbon or carbon-based particles include graphene quantum dots, (reduced) graphene oxide and carbon nanotubes.

Examples of particles comprising one or more light-absorbing compounds or particles loaded or functionalized with one or more light-absorbing compounds include particles loaded or functionalized with synthetic organic or inorganic absorbers as well as particles loaded or functionalized with naturally occurring absorbers or derivatives thereof. Particular examples comprise liposomes, solid lipid nanoparticles, polymer-based particles loaded or functionalized with light-absorbing dye molecules such as indocyanine green, inorganic quantum dots (having low fluorescence quantum yield), naturally occurring light absorbers like pigments (such as melanin, rhodopsin, photopsins or iodopsin) and synthetic analogs like polydopamine, or photosensitizers used in photodynamic therapy.

A preferred group of first particles comprises magnetic or magnetizable particles as, for example, iron oxide particles. Such particles have the advantage that they allow attraction, for example, to the biological barrier to be treated, for example, to the cell or cells to be treated, by means of a magnet or an array of magnets.

The first particle or first particles may also comprise a core surrounded with a shell. Examples of such core/shell particles comprise cores, such as polymer cores surrounded with a metal, a metal oxide, carbon, a carbon-based material, or one or more light-absorbing materials or compounds.

The core comprises, for example, polymeric particles such as polystyrene particles or poly(lactic-co-glycolic acid) particles, silica particles, hydrogels or vesicles that can be derived from biological cells or be synthetically created with lipids and/or polymers as primary constituents.

Examples of metals include gold, silver, platinum, palladium, copper and alloys thereof. Preferred metals comprise gold, silver and alloys thereof.

Examples of metal oxides include iron oxide, titanium oxide, zirconium oxide, cerium oxide, zinc oxide and magnesium oxide.

Examples of carbon or a carbon-based material comprise graphene or graphene oxide.

Examples of a light-absorbing compound comprises synthetic organic or inorganic absorbers as well as naturally occurring absorbers or derivatives thereof, for example, light-absorbing dye molecules such as indocyanine green, inorganic quantum dots (having low fluorescence quantum yield), naturally occurring light absorbers like pigments (such as melanin, rhodopsin, photopsins or iodopsin) and synthetic analogs like polydopamine, or photosensitizers used in photodynamic therapy.

As mentioned above, a vapor bubble can be generated by evaporation of the medium surrounding a nanobomb, in particular, the first particle(s) of a nanobomb and/or by (partial) evaporation of the first particle(s) or part of the first particle(s) of a nanobomb.

Preferred examples of first particles generating a vapor bubble by evaporation of the medium surrounding the first particle(s) comprise metal particles or metal oxide particles as well as carbon or carbon-based particles.

Preferred examples of first particles generating a vapor bubble by (partial) evaporation of the first particle or part of the first particle comprise particles comprising at least one constituent having a low boiling point such as liquids having a low boiling point enclosed or encapsulated in a matrix or surrounding shell. The matrix or surrounding shell further comprises one or more light-absorbing compounds. A particular example comprises particles comprising a low boiling point liquid such as perfluorocarbon or hexadecane (volatile oil) encapsulated with polymers or lipids into capsules further comprising one or more light-absorbing compounds such as an organic dye like Nile red. The dye can be incorporated in the matrix material, the shell, or can be dispersed, for example, in the oil phase. When irradiated with electromagnetic radiation, such particles induce localized heating and trigger the phase conversion of the encapsulated low boiling point liquid.

In preferred embodiments, the first particles can be functionalized, for example, to improve the interaction or connection with the second particle or particles, to attract the second particle or particles, to improve the interaction with the biological barrier, for example, the interaction with the cell or cells and/or to improve the stability of the first particle, the stability of the nanobombs and/or the stability of the composition. It is clear that the first particle can be functionalized in such a way to provide the first particle with more than one additional functionality.

Examples of functionalized first particles comprise first particles functionalized with one or more polymer, lipid and/or molecular linker to induce linking strategies such as bioconjugation (for example, with proteins, peptides, nucleic acids), complexation (for example, with molecular entities that can form complexes), or click chemistry (for example, with molecular entities that can form covalent bonds).

As second particles, any particle adapted to be propelled over a distance D away from the at least one first particle can be considered. Preferably, the second particles comprise biocompatible particles. More preferably, the second particles comprise clinically approved particles.

The second particles preferably have an average particle size between 10 nm and 10 μm or between 20 nm and 7 μm, more preferably between 50 nm and 5 μm or between 100 nm and 5 μm, as, for example, between 200 nm, 500 nm, 1 μm, 2 μm, 3 μm or 4 μm. As mentioned above, the average particle size is preferably determined using a high resolution imaging technique as, for example, transmission or scanning electron microscopy (TEM or SEM) or atomic force microscopy (AFM).

Preferred second particles comprise a polymer, a metal oxide, silicon or silicon oxide, liposome or liposomes or combinations thereof.

Preferred second particles comprise polymer particles (micro or nanoparticles), metal oxide particles (micro or nanoparticles), silicon or silicon oxide particles (micro or nanoparticles), liposomes, drug-loaded polymer particles, drug-loaded metal oxide particles, drug-loaded silicon or silicon oxide particles and drug-loaded liposomes.

Examples of polymer particles comprise polystyrene beads or poly(lactic-co-glycolic acid) (PLGA) beads.

Examples of metal oxide particles include titania or zirconia microparticles or nanoparticles.

Preferably, the second particles are adapted to create a minimum impact on nearby biological barriers, for example, on nearby cell membranes, once the second particles are propelled. Preferably, the second particles are propelled without detriment of their integrity to penetrate a biological barrier, for example, a cell membrane. More preferably, the second particles have a density that avoids rebound and allow either tube formation or localized deformation and penetration when propelled.

Preferably, the second particles are adapted to form pores in a biological barrier, for example, in a cell membrane. The pore size of the pores created by the second particles is preferably proportional to the size of the second particles. Preferably, the pore size is large enough to allow passage of the compounds to be delivered across the biological barrier, for example, across the cell membrane. The pore size preferably ranges between 1 nm and 5 μm, more preferably between 10 nm and 500 nm, or between 20 nm and 250 nm, for example, 50 nm, 100 nm or 150 nm.

Preferably, the second particle has a density of at least 1 kg/dm³, for example, a density ranging between 1 and 30 kg/dm³, for example, between 1 and 20 kg/dm³. More preferably, the second particle has a density of at least 5 kg/dm³ or a density of at least 10 kg/dm³, for example, a density ranging between 10 kg/m³ and 30 kg/dm³.

Preferably, the second particle has a size ranging between 1 nm and 5 μm and a density ranging between 1 and 30 kg/dm³. Preferred second particles have a particle size ranging between 10 nm and 500 nm and a density ranging between 1 and 10 kg/dm³.

In preferred embodiments, the at least one second particle can be functionalized, for example, to improve the interaction or connection with the first particle or first particles, to attract the first particle or first particles, to improve the interaction with the biological barrier, for example, with the cell or cells, to improve the stability of the second particle, to improve the stability of the nanobombs, to improve the stability of the composition or to provide the at least one second particle with one or more targeting moieties such as targeting antibodies, dyes or labels such as radiolabels. It is clear that the second particle can be functionalized in such a way as to provide the second particle with more than one additional functionality.

Examples of functionalized second particles comprise second particles functionalized with polymers or lipids, for example, to stabilize the particles or to allow electrostatic interactions, for example, with the first particle or particles and/or with the biological barrier; second particles loaded with drugs or other functional molecules such as targeting antibodies, dyes or labels, for example, radiolabels; and/or second particles functionalized with functional groups, for example, to induce a linking strategy such as bioconjugation (for example, with proteins, peptides, nucleic acids), complexation (for example, with molecular entities that can from complexes) and or click chemistry (for example, with molecular entities that can form covalent bonds) with a first particle.

Examples of polymers for functionalization are chitosan, poly(diallyldimethylammonium chloride), polyethylenimine, hyaluronic acid, poly(lactic-co-glycolic acid). Examples of lipids are 1,2-dioleoyl-sn-glycero-3-phosphoethanolamine (DOPE) and 1,2-dioleoyl-sn-glycero-3-succinate (DGS).

In particularly preferred embodiments, the compounds to be delivered across the biological barrier, for example, across the cell membrane, can be provided by the second particles themselves. In such preferred embodiments, the second particles are functionalized with the compound or compounds to be delivered across the biological barrier, for example, across the cell membrane. The second particles are, for example, functionalized with one or more functional moieties such as proteins, nucleic acids, drugs or labels such as radiolabels. The second particles are thereby functioning as nanoprojectiles to permeabilize or perforate the cell membrane and as a vehicle to deliver the compound or compounds directly into the cell or cells.

A preferred way of functionalization of the second particles with proteins, nucleic acids, polymers, drugs and/or labels is by chemical adsorption by electrostatic interaction, bioconjugation or chemical connection (click chemistry).

Another preferred way of functionalization of the second particles is by physical adsorption of charged molecules, for example, to improve the stability and/or electrostatic interaction with the first particle or first particles and/or with the second particle or second particles. Examples comprise adsorption of hyaluronic acid, polyethyleneimine (PEI) or polyethylene diallyl dimethyl amine hydrochloride (PDDAC).

A further preferred way of functionalization comprises physical adsorption of non-charged molecules, for example, to improve the stability. An example comprises adsorption of polyethylene glycol (PEG).

According to a second aspect of the present disclosure, a composition comprising at least one nanobomb, preferably comprising multiple nanobombs, as described above for use in a method to alter a biological barrier is provided. In particular, the composition is suitable to alter a biological barrier, for example, a cellular membrane and, more particularly, the composition is suitable to deform, permeabilize or perforate a biological barrier, for example, to deform, permeabilize or perforate a cellular membrane.

The method for altering, deforming or perforating a biological barrier, for example, a cellular membrane, preferably comprises the steps of:
providing a composition as described above comprising at least one nanobomb and preferably comprising multiple nanobombs, the at least one nanobomb or the multiple nanobombs comprising n first particles and m second particles;
introducing the composition in the proximity of a biological barrier, for example, in the proximity of cells,
irradiating the composition using electromagnetic radiation so as to form vapor bubbles, thereby generating a mechanical force to propel at least part of the m second particles of the nanobombs upon generation of the vapor bubbles, more particularly, upon expansion and/or collapse of the vapor bubbles.

The composition is preferably irradiated by a pulsed radiation source, although irradiation by a continuous wave radiation source can also be considered. The composition can be irradiated by one or more pulses.

When a pulsed radiation source is used, the pulses preferably have a duration in the range of 10 ns down to 0.1 ns or 10 fs.

The fluence (electromagnetic energy delivered per unit area) per pulse of the radiation source ranges preferably between 0.01 and 10 J/cm², more preferably between 0.05 and 2 J/cm² as, for example, 0.5 J/cm².

The wavelength of the radiation source may range from the ultraviolet region to the infrared region. In preferred methods, the wavelength range of the radiation used is in the visible to the near infrared region.

The composition comprising at least one nanobomb according to the present disclosure is, in particular, suitable for use in drug delivery, in intracellular delivery of compounds, in cell therapy, in immunotherapy, in gene therapy and in transfection of cells, for example, stem cells or T cells.

The composition comprising at least one nanobomb according to the disclosure is suitable for use in in vitro and ex vivo applications in which the nanobombs of the composition are brought in close proximity of the biological barrier, for example, by adding the composition to the cell medium. In case the nanobombs comprise magnetic or magnetizable particles, they can be concentrated toward the biological barrier, for example, to the cells, by means of an externally applied magnetic field.

It is clear that other configurations to bring the composition according to the present disclosure in close proximity of the biological barrier can be considered as well, for example, a configuration comprising nanobombs that are incorporated on top of or embedded in a substrate onto which cells can be cultured.

In particular, the composition is suitable for use in intracellular delivery of nucleic acids, including oligonucleotides, siRNA, mRNA or pDNA.

The composition is also suitable for use in the intracellular delivery of nucleoproteins, including ribonucleoproteins, such as Cas9/gRNA.

Furthermore, the composition is suitable for use in the intracellular delivery of peptides and proteins, such as nanobodies or antibodies.

In addition, the composition is suitable for use in the intracellular delivery of contrast agents, such as quantum dots, iron oxide nanoparticles and gadolinium chelates.

The composition is furthermore suitable for use in the intracellular delivery of plasmonic nanoparticles, for example, for sensing and characterization purposes as, for example, LSPR sensors (localized surface plasmon resonance) or for SERS (surface-enhanced raman spectroscopy).

The composition is furthermore suitable for use in in vivo applications. In case the nanobombs comprise magnetic or magnetizable particles, it is possible to preferentially localize the nanobombs in a particular body region by using a magnet or magnets.

According to a third aspect of the disclosure, an ex vivo or in vitro method for altering a biological barrier is provided. The method comprises the steps of:
providing a composition comprising nanobombs comprising n first particles and m second particles as described above;
introducing the composition in the proximity of a biological barrier, for example, in the proximity of cells,
irradiating the composition using electromagnetic radiation so as to form vapor bubbles, thereby generating a mechanical force to propel at least part of the second particles of the nanobombs upon generation of the vapor bubbles, more particularly, upon expansion and/or collapse of the vapor bubbles.

Preferred methods comprise methods to deform, permeabilize or perforate a biological barrier, such as methods to deform, permeabilize or perforate a cellular membrane.

The method may further comprise the step of attracting the nanobombs of the composition and, in particular, the first particle or first particles of the nanobombs to the biological barrier by means of a magnetic field.

According to a fourth aspect of the disclosure, a method to prepare a composition comprising at least one nanobomb, preferably comprising multiple nanobombs, as described above is provided. Any method that allows obtaining such composition can be considered.

A preferred method to prepare a composition comprising at least one nanobomb comprises the steps of:
providing first particles able to absorb electromagnetic radiation so as to generate a vapor bubble;
providing second particles; and
mixing the first particles and the second particles allowing formation of at least one nanobomb comprising n first particles and m second particle, with each of n and m being at least one and with at least one of the m second particles being in close proximity to at least one of the n first particles, with “being in close proximity to” being defined as being either in contact with or being positioned at a distance d smaller than 1 μm.

In preferred methods, the first particles, the second particles or the first and second particles are functionalized, preferably before being mixed. Any method known in the art can be considered to functionalize the first particles and/or the second particles.

A particularly preferred method to prepare a composition according to the present disclosure comprises the mixing of first particles and second particles under rotation, for example, 24 hours of rotation, to allow self-assembling.

The method to prepare the composition may further comprise one or more additional steps as, for example, one or more purification steps after the mixing of the first and second particle(s) and/or between two mixing steps.

As a purification step, any purification method known in the art can be considered. Examples comprise washing, magnetic washing, filtering, centrifugation, etc.

An alternative method to prepare a composition according to the disclosure comprises depositing at least one first particle into a cavity or protrusion of a container or platform, followed by depositing or attaching at least one second particle. The cavity or protrusion can be either functionalized or non-functionalized.

### BRIEF DESCRIPTION OF THE DRAWINGS

The present disclosure will be discussed in more detail below, with reference to the accompanying drawings, in which:

FIG. 1 illustrates the synthesis of a nanobomb and the irradiation of such nanobomb followed by the generation of a vapor bubble to propel the second particles of a nanobomb, thereby inducing pore formation in a biological barrier;

FIG. 2 illustrates a first type of a nanobomb according to the present disclosure comprising a first particle surrounded by one layer of second particles;

FIG. 3 illustrates a second type of a nanobomb according to the present disclosure comprising a container with first and second particles;

FIGS. 4A and 4B show dark field microscopy images of nanobombs before and immediately after irradiation with pulsed laser light;

FIG. 5 shows a confocal image of Hela cells and nanobombs according to the present disclosure after laser irradiation;

FIGS. 6A and 6B compare confocal images of Hela cells using nanobombs comprising first and second particles and using first and second particles (uncoupled);

FIG. 7 illustrates the efficiency of transfection with mRNA using traditional photoporation with gold nanoparticles and using nanobombs according to the present disclosure;

FIG. 8 shows the size (bars) and zeta potential (black dots) of different first particles, second particles and nanobombs according to the present disclosure determined by Dynamic Light Scattering (DLS) and Scanning Electron Microscopy (SEM) (for the case of nanobomb s);

FIG. 9 shows the effect of laser fluence on the number of generated vapor nanobubbles;

FIG. 10 shows the transfection efficiency of FITC dextran FD500 (500 kDa) in Hela cells using different types of nanobombs having second particles of increasing mass density;

FIG. 11 shows the efficiency of transfection (in transfected cells) with mRNA in Hela cells as well as the cell viability (in %) of a method according to the present disclosure using nanobombs having 200 nm PLGA nanoparticles as second particles, compared to non-transfected cells and compared to traditional photoporation;

FIG. 12 shows the efficiency of transfection (in transfected cells) with mRNA in Jurkat cells as well as the cell viability (in %) using a method according to the present disclosure using nanobombs having 200 nm PLGA nanoparticles as second particles, compared to non-transfected cells and compared with photoporation.

### DETAILED DESCRIPTION

The disclosure will be described with respect to particular embodiments and with reference to certain drawings but the disclosure is not limited thereto but only by the claims. The drawings are only schematic and are non-limiting. The size of some of the elements in the drawings may be exaggerated and not drawn on scale for illustrative purposes. The dimensions and the relative dimensions do not correspond to actual reductions to practice of the disclosure.

When referring to the endpoints of a range, the endpoint values of the range are included.

When describing the disclosure, the terms used are construed in accordance with the following definitions, unless indicated otherwise.

The terms “first,” “second,” and the like, used in the description as well as in the claims, are used to distinguish between similar elements and not necessarily describe a sequence, either temporally, spatially, in ranking or in any other manner. It is to be understood that the terms so used are interchangeable under appropriate circumstances and that the embodiments of the disclosure described herein are capable of operation in other sequences than described or illustrated herein.

The term “and/or” when listing two or more items, means that any one of the listed items can by employed by itself or that any combination of two or more of the listed items can be employed.

The term “generation of a vapor bubble” includes either expansion of the vapor bubble, either collapse of the vapor bubble or a combination of expansion and collapse of the vapor bubble and secondary effects that can be the result of the bubble expansion and collapse, such as pressure waves and flow of the surrounding medium.

The term “microparticle” refers to particles having a diameter or equivalent diameter ranging between 1 μm and 100 μm. The term “nanoparticle” refers to particles having a diameter or equivalent diameter ranging between 1 nm and 1000 nm.

The term “vapor bubble” or “bubble” refers to vapor nanobubbles and vapor microbubbles. Preferably, the term “vapor bubble” or “bubble” refers to vapor bubbles having a diameter in the range of 10 nm to 100 μm. Vapor bubbles comprise water vapor bubbles, although embodiments are not limited thereto.

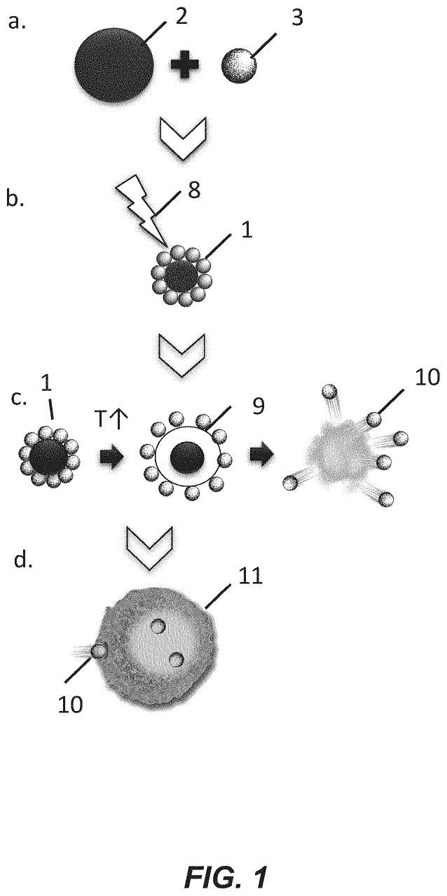

FIG. 1 illustrates the synthesis of a nanobomb (step a) as well as the irradiation of such nanobombs (step b), followed by the generation of the vapor bubble (step c) and the perforation of a cell by the propelled second particles of nanobombs (step d).

Nanobombs 1 are synthesized by mixing first particles 2 (acting as vapor nanobubble source) and second particles 3 (acting as nanoprojectiles) (step a). The nanobombs 1 are irradiated, preferably using short pulsed laser light 8 of sufficient intensity (step b). Upon irradiation, the nanobombs 1 and, in particular, the first particles 2 of the nanobombs 1 heat up. The temperature exceeds the surrounding medium's boiling temperature, thereby evaporating the surrounding medium and forming vapor bubbles 9. Alternatively or additionally, the temperature exceeds the evaporation temperature of the first particle 2 or of part of the first particle 2 and the first particle 2 or part of the first particle 2 evaporates forming vapor bubbles 9. The vapor bubbles 9 are quickly expanding around the first particles 2. The expanding, and possibly the collapsing, of the vapor bubble 9 causes that the second particles 3 are propelled away from the first particle (step c). The propelled second particles are indicated by reference number 10. The propelled second particles 10 may cause pore formation in the membrane of a nearby cell 11 (step d).

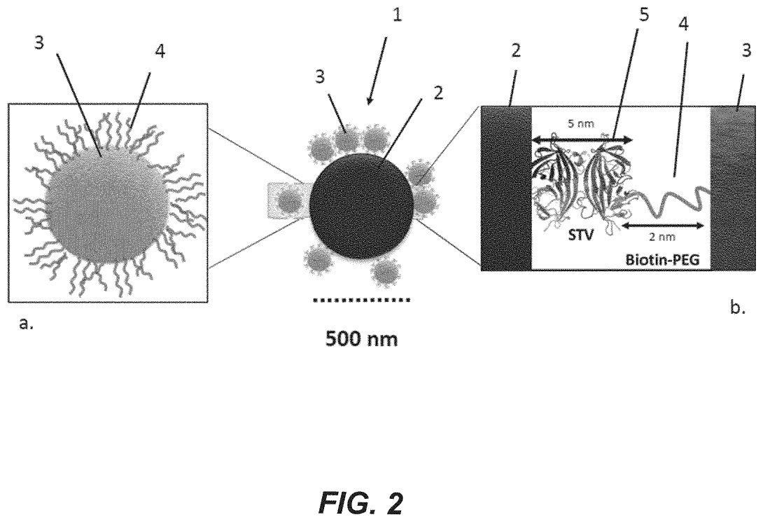

FIG. 2 illustrates a first type of a nanobomb 1 according to the present disclosure. The nanobomb 1 comprises an iron oxide nanoparticle (IONP) as first particle 2 and fluorescent polystyrene nanospheres as second particles 3. The IONP has, for example, an average diameter of 500 nm and may generate a vapor bubble upon irradiation with a single laser pulse, for example, a 7 ns laser pulse with a fluence of 1 J/cm² and a wavelength of 561 nm. The IONP can be functionalized with streptavidin molecules 5. A detail of a second particle 3 is given in box a of FIG. 2. The second particles 3 comprise fluorescent nanospheres having an average diameter of 100 nm and functionalized with biotin 4. The second particles 3 are, for example, attached to the first particle 1 by biotin-streptavidin linker moieties 4 as shown in box b of FIG. 2.

It is clear for a person skilled in the art that other linking strategies such as bioconjugation, complexation, electrostatic connection, physisorption, chemical connection, for example, by one or more covalent bonds (click chemistry) can also be considered.

Furthermore, it is clear for a person skilled in the art that a nanobomb according to the disclosure may comprise more than one first particle, for example, in contact with or connected to each other such as by bioconjugation, complexation, electrostatic connection, physisorption, or chemical connection.

It is also clear for a person skilled in the art that a nanobomb according to the disclosure may comprise a first particle or a plurality of first particles surrounded by more than one layer of second particles, for example, surrounded by two or three layers of second particles.

Table 1 mentions further examples of nanobombs according to the present disclosure by specifying the type of the first particle (functioning as vapor nanobubble (VNB) source), the surface functionalization of the first particle, the type of the second particle (functioning as projectile), the surface functionalization of the second particle and the linking strategy between the first particle(s) and the second particle(s).

TABLE 1

| | Surface | Second | Surface | |
|---|---|---|---|---|
| First particle | functionalization | particle | functionalization | Linking |
| (VNB source) | of first particle | (Projectile) | of second particle | strategy |
| Iron oxide | Coating or surface | Polystyrene | Coating or surface | Covalent bond |
| Gold | ligands exposing - | Polymeric NP | ligands exposing - | formation |
| Titanium oxide | NH₂ groups | Polyplexes | COOH groups | through |
| Carbon | | Liposomes | | Carbodiimide |
| Nanotubes | | Silica | | Crosslinker |
| Graphene oxide | | Titanium oxide | | Chemistry |
| Polydopamine | | | | |
| Poly(N- | | | | |
| phenylglycine) | | | | |
| Iron oxide | Coating or surface | Polystyrene | Coating or surface | Covalent bond |
| Gold | ligands exposing - | Polymeric NP | ligands exposing - | formation |
| Titanium oxide | COOH groups | Polyplexes | NH₂ groups | through |
| Carbon | | Liposomes | | Carbodiimide |
| Nanotubes | | Silica | | Crosslinker |
| Graphene oxide | | Titanium oxide | | Chemistry |
| Polydopamine | | | | |
| Poly(N- | | | | |
| phenylglycine) | | | | |
| Iron oxide | Coating or surface | Polystyrene | Coating or surface | Covalent bond |
| Gold | ligands exposing - | Polymeric NP | ligands exposing | formation |
| Titanium oxide | N₃ groups | Polyplexes | propargyl groups | catalyzed by |
| Carbon | | Liposomes | | Cu⁺ |
| Nanotubes | | Silica | | |
| Graphene oxide | | Titanium oxide | | |
| Polydopamine | | | | |
| Poly(N- | | | | |
| phenylglycine) | | | | |
| NP | | | | |
| Iron oxide | Coating or surface | Polystyrene | Coating or surface | Covalent bond |
| Gold | ligands exposing | Polymeric NP | ligands exposing - | formation |
| Titanium oxide | propargyl groups | Polyplexes | N₃ groups | catalyzed by |
| Carbon | | Liposomes | | Cu⁺ |
| Nanotubes | | Silica | | |
| Graphene oxide | | Titanium oxide | | |
| Polydopamine | | | | |
| Poly(N- | | | | |
| phenylglycine) | | | | |
| Iron oxide | Coating or surface | Polystyrene | Coating or surface | electrostatic |
| Gold | ligands exposing | Polymeric NP | ligands exposing | |
| Titanium oxide | positively charged | Polyplexes | negatively charged | |
| Carbon | groups | Liposomes | groups | |
| Nanotubes | | Silica | | |
| Graphene oxide | | Titanium oxide | | |
| Polydopamine | | | | |
| Poly(N- | | | | |
| phenylglycine) | | | | |
| Iron oxide | Coating or surface | Polystyrene | Coating or surface | electrostatic |
| Gold | ligands exposing | Polymeric NP | ligands exposing | |
| Titanium oxide | negatively charged | Polyplexes | positively charged | |
| Carbon | groups | Liposomes | groups | |
| Nanotubes | | Silica | | |
| Graphene oxide | | Titanium oxide | | |
| Polydopamine | | | | |
| Poly(N- | | | | |
| phenylglycine) | | | | |
| Iron oxide | Coating or surface | Polystyrene | Coating or surface | bioconjugation |
| Gold | ligands exposing | Polymeric NP | ligands exposing | |
| Titanium oxide | proteins | Polyplexes | specific ligands | |
| Carbon | (e.g., streptavidin, | Liposomes | (e.g., biotin, | |
| Nanotubes | antibody, | Silica | antigens, etc.) | |
| Graphene oxide | nanobody, etc.) | Titanium oxide | | |
| Polydopamine | | | | |
| Poly(N- | | | | |
| phenylglycine) | | | | |
| Iron oxide | Coating or surface | Polystyrene | Coating or surface | bioconjugation |
| Gold | ligands exposing | Polymeric NP | ligands exposing | |
| Titanium oxide | specific ligands | Polyplexes | proteins | |
| Carbon | (e.g., biotin, | Liposomes | (e.g., streptavidin, | |
| Nanotubes | antigens, etc.) | Silica | antibody, | |
| Graphene oxide | | Titanium oxide | nanobody, etc.) | |
| Polydopamine | | | | |
| Poly(N- | | | | |
| phenylglycine) | | | | |
| Iron oxide | DNA strand | Polystyrene | Complementary | bioconjugation |
| Gold | | Polymeric NP | DNA strand | |
| Titanium oxide | | Polyplexes | | |
| Carbon | | Liposomes | | |
| Nanotubes | | Silica | | |
| Graphene oxide | | Titanium oxide | | |
| Polydopamine | | | | |
| Poly(N- | | | | |
| phenylglycine) | | | | |

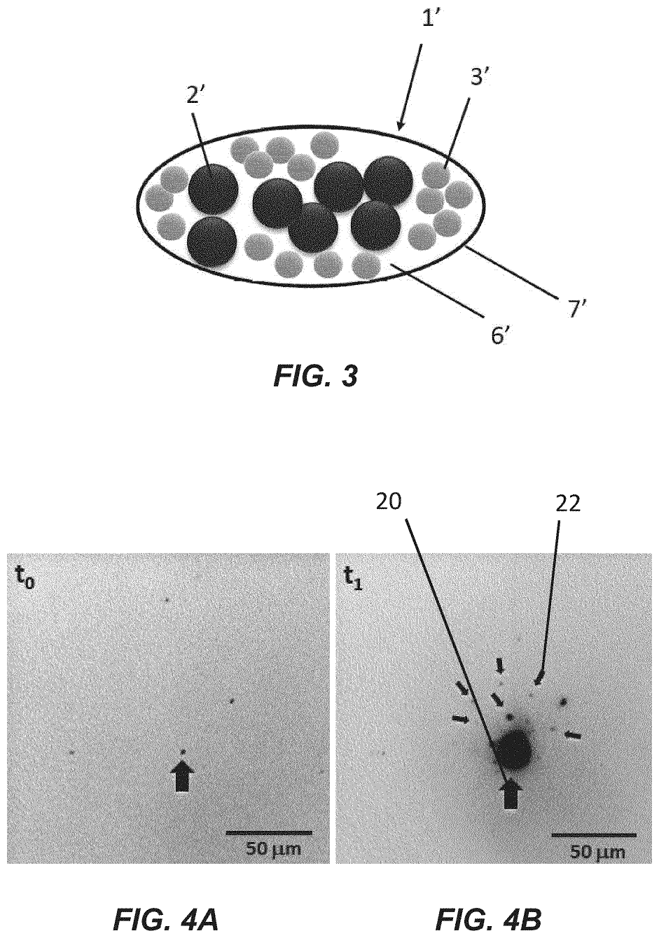

FIG. 3 illustrates a further embodiment of a nanobomb 1′ according to the disclosure. The nanobomb 1′ comprises first particles 2′ and second particles 3′ held together by a matrix 6′ or a shell 7′. It is clear that the matrix material 6′ or the shell 7′ may comprise one nanobomb or a plurality of nanobombs.

FIGS. 4A and 4B illustrate the optical triggering of a nanobomb as visualized with dark field microscopy. FIG. 4A shows a dark field microscopy image of a dispersion of nanobombs in water as illustrated in FIG. 1 before irradiation (at time to) and FIG. 4B shows a dark field microscopy image right after irradiation of the nanobomb indicated by the arrow in FIG. 4A with a single 7 ns laser pulse (at time ti). FIG. 4B clearly illustrates the generation of the vapor bubble from the first particle of the nanobomb (indicated by arrow 20) as well as the propelling of the second particles (indicated by arrows 22). The second particles 22 are thereby propelled over tens of micrometers in the surrounding medium.

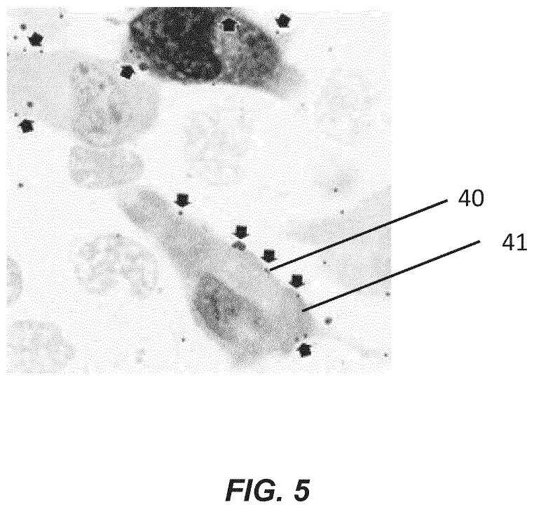

Penetration of nanospheres of a nanobomb into cells after irradiation of a nanobomb could be demonstrated by confocal images. To demonstrate penetration of the nanospheres into the cells' nanobombs according to the disclosure, in particular, nanobombs as illustrated in FIG. 1, are added to cultured cells, together with Propidium Iodide (PI), as a marker for membrane permeabilization. The confocal image of FIG. 5 shows that after laser irradiation, the nanospheres had successfully penetrated into the cells with a concomitant influx of PI into the cell's cytoplasm. The second particles 40 are found partially in the cells 41.

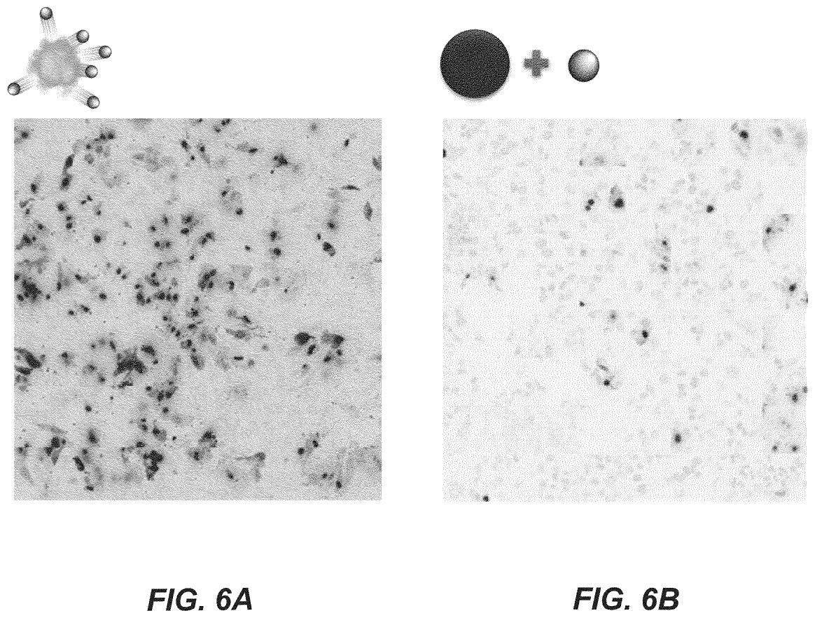

FIGS. 6A and 6B show confocal images of cultured cells together with Propidium Iodide as marker in the presence of nanobombs according to the present disclosure, i.e., nanobombs comprising first and second particles (FIG. 6A) and in the presence of the uncoupled first and second particles (FIG. 6B). As shown in FIGS. 6A and 6B, PI could be delivered into most cells using the nanobombs according to the disclosure, while this was clearly not the case in the control experiment where IONP and nanospheres were added to the cells as a mixture of both components, i.e., without the first and second particles being in contact to one another to form the actual nanobombs.

Transfection of cells with mRNA encoding for GFP using traditional photoporation was compared with transfection of cells with mRNA using nanobombs as illustrated in FIG. 1.

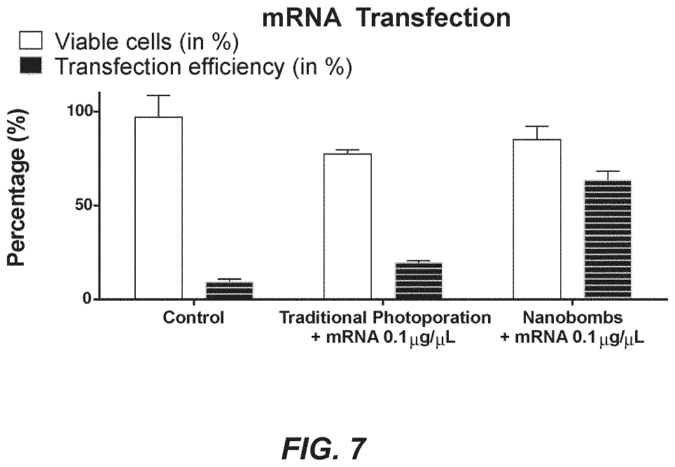

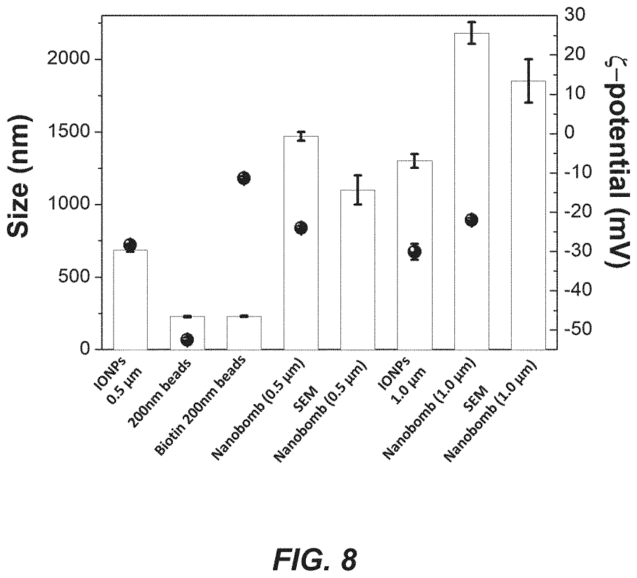

The traditional photoporation was performed by 30 minutes incubation with 70 nm gold nanoparticles (AuNPs) (8.5×10⁷ AuNPs/mL), positively charged. After this period, the AuNPs are washed and medium containing mRNA was added. The cells were immediately irradiated. For the transfection using nanobombs according to the disclosure, a mixture of the nanobombs (6.4×10⁸ nanobombs/mL) and the mRNA in medium was added to the cells and incubated for 5 minutes before laser treatment. For both methods, the cells were washed and new medium was added after the laser treatment and the green fluorescence protein (GFP) was checked after 24 hours. For each experiment, 15,000 cells were seeded in 96-well plates 24 hours prior to the experiment. The results are shown in FIG. 7. The efficiency of transfection with mRNA using nanobombs according to the disclosure is considerably higher than the efficiency with mRNA using traditional photoporation: 70% transfected cells vs. 20% transfected cells without additional cytotoxicity.

FIG. 8 shows the size (bars) and zeta potential (black dots) of different first particles, second particles and nanobombs according to the disclosure determined by DLS (Dynamic Light Scattering) and SEM (Scanning Electron Microscopy (for the case of nanobombs). The first particles, the second particles and the nanobombs that are considered are:
iron oxide nanoparticles (IONPs) having a diameter of 0.5 μm;
polystyrene beads having a diameter of 200 nm;
polystyrene beads having a diameter of 200 nm functionalized with Biotin;
nanobombs comprising a core of IONP having a diameter of 0.5 μm surrounded with an average of 35 polystyrene beads having a diameter of 200 nm functionalized with Biotin;
iron oxide nanoparticles (IONPs) having a diameter of 1 μm;
nanobombs comprising a core of IONP having a diameter of 1 μm surrounded with polystyrene beads having a diameter of 200 nm functionalized with Biotin.

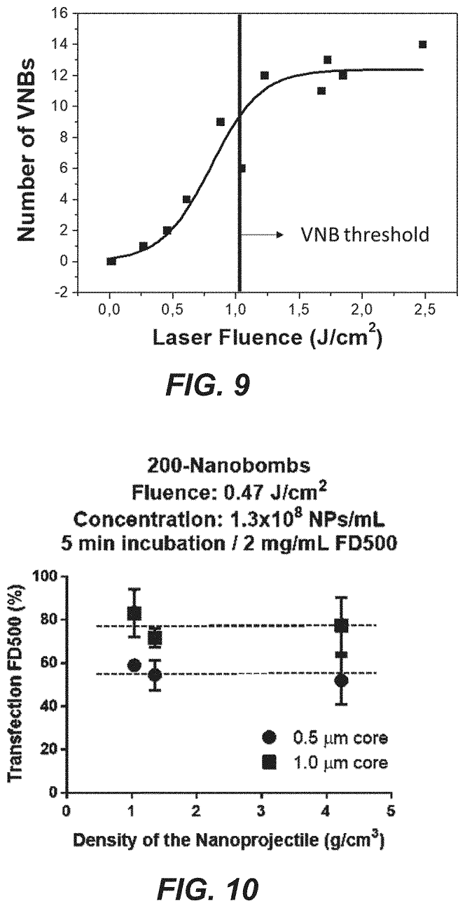

FIG. 9 shows the effect of laser fluence on the number of generated vapor nanobubbles using nanobombs comprising a core of 0.5 μm IONP surrounded by 200 nm polystyrene beads. The fluence threshold (90% probability) was determined to be 1.05 J/cm².

FIG. 10 shows the delivery efficiency of FITC dextran FD500 (500 kDa) in Hela cells using different nanobombs having either a core of 0.5 μm or a core of 1 μm. The nanobombs (with a core of 0.5 μm and with a core of 1 μm) have second particles (nanoprojectiles) of 200 nm of one of the following materials:
polystyrene: density of 1.04 g/cm³
PLGA (poly(lactic-co-glycolic acid): density 1.37 g/cm³
TiO₂: density of 4.30 g/cm³

PLGA has the advantage of being a biodegradable, biocompatible and FDA- and EMA-approved material.

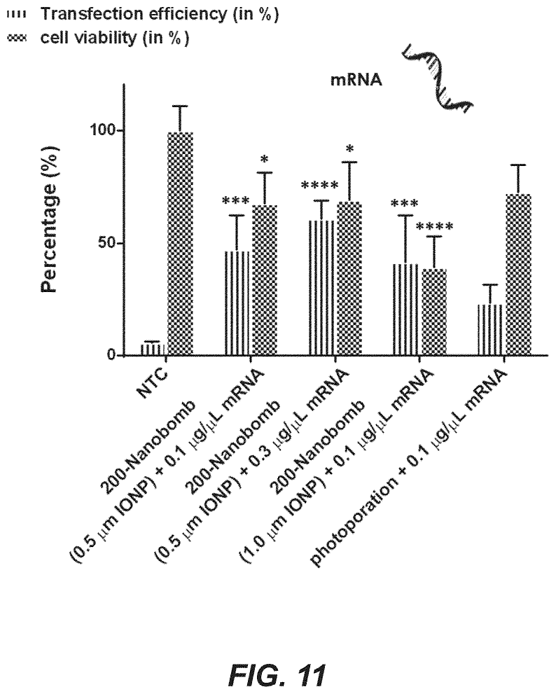

FIG. 11 shows the efficiency of transfection (in %) with mRNA in Hela cells as well as the cell viability (in %) using a method according to the disclosure compared with non-transfected cells and compared with photoporation. The nanobombs were irradiated with a single laser pulse at the previously determined fluence threshold using 1.3×10⁸ nanobombs/mL with an incubation time of 5 minutes. For photoporation, a concentration of 4×10⁷ gold nanoparticles/mL was used.

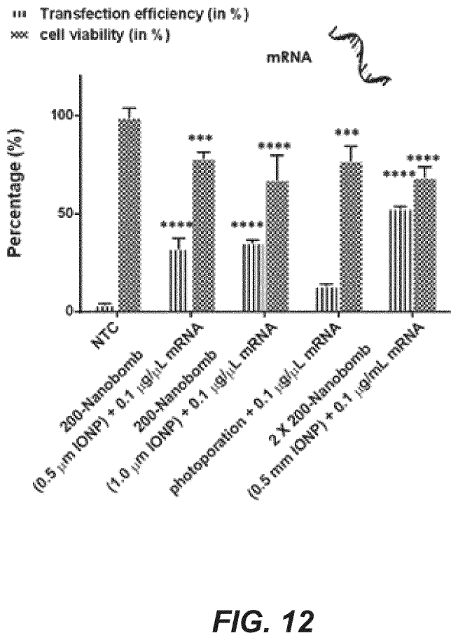

FIG. 12 shows the efficiency of transfection (in %) with mRNA in Jurkat cells as well as the cell viability (in %) using a method according to the disclosure compared to non-transfected cells and compared with photoporation. The nanobombs were irradiated with a single laser pulse at the previously determined fluence threshold using 1.3×10⁸ nanobombs/mL with an incubation time of 20 minutes. For photoporation, a concentration of 4×10⁷ gold nanoparticles/mL was used.

1. A composition comprising at least one nanobomb, said at least one nanobomb comprising n first particles and m second particles, with each of n and m being at least one, at least one of said m second particles being in close proximity to at least one of said n first particles, so as to either be in contact with or be positioned at a distance d smaller than 1 μm, said at least one n first particle being able to absorb electromagnetic radiation such so as to generate a vapor bubble, whereby said generation of said vapor bubble causes said at least one m second particle to be propelled over a distance D away from said at least one n first particle, with said distance D being at least 0.01 μm.

2. The composition according to claim 1, wherein said at least one m second particle of said at least one nanobomb is adapted to alter a biological barrier once propelled upon said generation of said vapor bubble.

3. The composition of claim 1, wherein m is larger than n.

4. The composition of claim 1, wherein said m second particle(s) of said at least one nanobomb has a size ranging between 10 nm and 10 μm and/or a density of at least 1 kg/dm³.

5. The composition of claim 1, wherein a majority of said n first particles comprises at least p second particles in close proximity, with p being at least 2.

6. The composition of claim 1, wherein said distance D ranges between 0.1 and 100 μm.

7. The composition of claim 1, wherein said at least one n first particle comprises a metal, a metal oxide, carbon, a carbon-based material, a light-absorbing compound or particles loaded or functionalized with one or more light-absorbing compounds or a combination thereof.

8. The composition of claim 1, wherein said at least one n first particle is functionalized with one or more polymer, lipid and/or molecular linker.

9. The composition of claim 1, wherein said m second particle(s) of said at least one nanobomb is selected from the group consisting of polymer particles, metal oxide particle, silicon or silicon oxide particles, liposomes, drug loaded polymer particles, drug loaded silicon or silicon oxide particles an drug loaded liposomes.

10. The composition of claim 1, wherein said at least one m second particle is functionalized with one or more charged polymer or lipid, one or more targeting moiety selected from the group consisting of antibodies, dyes, proteins, nucleic acids, drugs and/or labels and/or one or more functional group to induce a linking strategy with the at least one n first particle in close proximity.

11. A method of altering a biological barrier, the method comprising: using the composition of claim 1 to alter at least one biological barrier.

12. The composition of claim 1 for use in drug delivery, in intracellular delivery of compounds, in drug delivery, in cell therapy, in immunotherapy, in gene therapy and in transfection of cells.

13. An ex vivo or in vitro method for altering a biological barrier, said method comprising:
providing the composition of claim 1;
introducing the composition in proximity of a biological barrier; and
irradiating the composition using electromagnetic radiation so as to generate vapor bubbles, thereby generating a mechanical force to propel at least part of said m second particles of said at least one nanobomb of said composition upon the generation of said vapor bubbles.

14. The method of claim 13, further comprising attracting said at least one nanobomb of said composition to said biological barrier by means of a magnetic field.

15. A method of producing the composition of claim 1, the method comprising:
providing first particles able to absorb electromagnetic radiation so as to generate a vapor bubble,
providing second particles; and
mixing said first particles and said second particles allowing to form at least one nanobomb, said at least one nanobomb comprising n first particles and m second particles, with each of n and m being at least one and with at least one of said m second particles being in close proximity to at least one of said n first particles, with “being in close proximity to” being defined as being either in contact with or being positioned at a distance d smaller than 1 μm.

16. A composition comprising a nanobomb, wherein the nanobomb has n first particles and m second particles, wherein each of n and m is at least one, and wherein at least one of the m second particles is in close proximity to at least one of the n first particles, so as to either be in contact with one another or to be positioned at a distance d of less than 1 μm from one another, wherein the at least one n first particle absorbs electromagnetic radiation and thereby generates a vapor bubble, wherein generation of a vapor bubble causes said at least one m second particle to be propelled over a distance D away from said at least one n first particle, wherein distance D is at least 0.01 μm.

17. The composition of claim 16, wherein m is greater than n.

18. The composition of claim 17, wherein the at least one m second particle has a size of between 10 nm and 10 μm and/or a density of at least 1 kg/dm3.

19. The composition of claim 18, wherein distance D is between 0.1 and 100 μm.

20. The composition of claim 19, wherein the at least one n first particle comprises a metal, a metal oxide, carbon, a carbon-based material, a light-absorbing compound, a particle loaded or functionalized with one or more light-absorbing compounds, or a combination of any thereof.
